(19) United States
(12) Patent Application Publication
(10) Pub. No.: US 2002/0143189 A1
(43) Pub. Date: Oct. 3, 2002

(54) Potassium dinitro benzofuroxane and method of making same

(76) Inventor: Chiranjeevirao Sonti, Newark, DE (US)

Correspondence Address: Charles E. Temko, Temko & Temko, 22 Marion Road, Westport, CT, 06880, US

(21) Appl. No.: 09/822,084

(22) Filed: Apr. 2, 2001

(51) Int. Cl. C07D 271/12

(52) U.S. Cl. 548/126

(57) ABSTRACT

Potassium dinitro benzofuroxane and method of making same. The combination is useful as a coating on an electroconductive wire forming part of a squib for commencing the inflation of an automotive safety airbag, as well as other uses.

### BACKGROUND OF THE INVENTION

This invention relates generally to the field of organic chemistry, and more particularly to a chemical composition used in the manufacture of squibs employed in the manufacture of automotive safety restraint systems, typically driver and passenger airbags. The squib will include a fine metallic wire upon which a very small quantity of a chemical composition is coated. On passing a few milliamperes of current through the metallic wire, a chemical reaction takes place and energy will be liberated in the form of a spark which will trigger a set of reactions, ultimately liberating a cushion of air which inflates the bag at either side of an automobile.

In the prior art, the wire is typically coated with lead styphanate, which lacks the degree of reliability necessary in an airbag system. In addition, because this substance contains a poisonous metal, its use is not acceptable for environmental protection agency regulations. There thus has arisen a need for an alternative material which may substitute lead styphanate. The present disclosure is directed to the making of such substitute, potassium dinitro benzofuroxane (KDNBF).

### SUMMARY OF THE INVENTION

Briefly stated, the invention contemplates the synthesis of KDNBF using benzofuroxane, fuming nitric acid, sulfuric acid, and potassium bicarbonate. Because of the high concentrations of sulfuric and nitric acids, the reactions must be performed during carefully regulated conditions. The resultant material is a crystalline solid which can be stored under dry conditions prior to use, and may be applied by mixing the same with any of a number of suitable evaporable vehicles.

### DETAILED DESCRIPTION OF THE DISCLOSED EMBODIMENT

The following example describes preparation of KDNBF in laboratory conditions. It will be apparent to those skilled in the art that larger quantities can be prepared under similar conditions for commercial use.

### EXAMPLE 1

300 ml of concentrated sulfuric acid is placed in a one liter stainless steel container, following which the container is placed in a larger container, and the space between is filled with crushed ice. The liquid in the stainless steel container is stirred continuously until the same attains a temperature range of 0 to 5 C.

To the acid, in small increments, there is added benzofuroxane accompanied by constant stirring, again taking care to maintain the temperature of the solution at or below five degrees C. The weight of the solid benzofuroxane is 50 grams, and all of the added solid will be dissolved to result in a brown liquid.

Next, place 200 mi of 98 percent pure and concentrated sulfuric acid in a one liter glass beeker, maintaining this container in an ice bath until the temperature of the acid is in the range of 0-5 degrees C. While the container is in the ice bath, there is slowly added with constant stirring, 100 ml of equally cooled (0-5 C.) fuming nitric acid taking care that the temperature of the acid mixture does not increase. The resulting acid mixture may be referred to as a nitration mixture.

The cooled nitration mixture is poured into a 500 ml separating funnel, which is added in small increments to the container containing the benzofuroxane solution, with constant stirring.

Next, the stainless steel container containing the reaction mixture is placed on a hot plate and heated with constant stirring to a temperature of approximately 40 degrees C.

Using a six liter stainless steel vessel, the contents of the one liter stainless steel container is poured very slowly and carefully to form a yellow colored dinitro benzofuroxane, together with small amounts of unreacted nitration mixer.

Using a vacuum filtration filter, the contents of the six liter vessel are separated, and the dinitro benzofuroxane solid on the filter paper is washed with distilled water until it is free of any unreacted acid.

Using another one liter glass beaker, 300 ml of concentrated nitric acid is added to the dinitro benzofuroxane, and this mixture is maintained on a hot plate to bring the contents to about 60-70 degrees C. with constant stirring, so that all of the pure dinitro benzofuroxane in the mixer will be dissolved in hot nitric acid. The hot mixture is then filtered to separate any insoluble substances.

The clear liquid is then cooled to about 15-20 degrees C. in an ice bath with constant stirring causing pure dinitro benzofuroxane to crystallize and precipitate. The precipitate is then vacuum filtered, and washed on the filter paper several times to assure freedom from unreacted acid

Next, a one liter glass beaker is filled with about 500 ml distilled water, and the now pure dinitro benzofuroxane is added, following which the contents are heated to about 75 degrees C. using a hot plate, and at this temperature level, there is slowly added solid potassium bicarbonate. With each addition of the bicarbonate, a further chemical reaction will take place liberating CO₂. The potassium bicarbonate is continued until there is no further liberation of CO₂. The resulting product will be potassium dinitro benzofuroxane.

To complete the procedure, the contents of the beaker are cooled to room temperature, and the precipitate is vacuum filtered and washed with ethyl alcohol. This material is then dried at approximately 40 degrees C. under vacuum in a hot air oven. The purity of the material may be proven with a differential calorimeter (DSC) which will also provide both the melting point of the material and the calorific value.

I wish it to be understood that I do not consider the invention to be limited to the precise details disclosed in the specification, for obvious modifications will occur to those skilled in the art to which the invention pertains.

I claim:

1. As a new composition of matter, potassium dinitro benzofuroxane.

2. The reaction product of benzofuroxane, nitric acid, sulfuric acid, and potassium bicarbonate in accordance with the following equations:
C₆H₄N₂O₂+2HNO₃→C₆H₂(NO₂)₂N₂O₂+2H₂O Dinitro Benzofuroxane
C₆H₂(NO₂)₂N₂O₂+2KHCO₃→KC₆H(NO₂)₂N₂O₂+H₂O+C₆ Potassium Dinitro Benzofuroxane

3. The method of making potassium benzofuroxane comprising the steps of:
a) reacting benzofuroxane with sulfuric acid;
b) reacting the product of step (a) with a nitration mixer of nitric acid and sulfuric acid to form dinitro benzofuroxane, and;
c) reacting the product of step (b) with potassium bicarbonate.
